(19) United States
(12) Patent Application Publication
(10) Pub. No.: US 2016/0128752 A1
(43) Pub. Date: May 12, 2016

(54) MIXING AND DISCHARGING DEVICE HAVING A THREADED SPINDLE

(71) Applicant: MEDMIX SYSTEMS AG, Rotkreuz (CH)

(72) Inventors: Andy GRETER, Rotkreuz (CH); Beat MATHYS, Muri (CH)

(73) Assignee: Medmix Systems AG, Rotkreuz (CH)

(21) Appl. No.: 14/898,324

(22) PCT Filed: May 27, 2014
(86) PCT No.: PCT/EP2014/060942

§ 371 Date: Dec. 14, 2015

(30) Foreign Application Priority Data
Jun. 18, 2013 (CH) 1138/13

(51) Int. Cl. A61B 17/88; B01F 7/00; B01F 15/02

(52) U.S. Cl. CPC A61B 17/8822; A61B 17/8825; B01F 15/0237; B01F 7/00391; A61B 2017/883

(57) ABSTRACT

A mixing and discharging device (1) has a syringe-like container (10), in which a plunger can be moved. A press-out spindle (30) having an external thread (31) interacts with a counterpart (40) by means of a thread engagement. The counterpart comprises one or more thread segments, which can be deflected outward in order to release the thread engagement. A securing element (5) can be slid onto the counterpart (40) in a distal direction in order to prevent a release of the thread engagement. A mixing rod (61) extends through the plunger (20). An operating element (70) is connected to the proximal end of the mixing rod by means of a releasable form closure or force closure. A locking element (80) can be moved between a locking position and a release position. In the locking position, the locking clement prevents a release of the form closure or force closure. Insulating ribs (13) minimise the input of heat into the interior of the container due to touching.

### TECHNICAL SCOPE

The present invention relates to a device for discharging a product. In particular, the product can be a bone cement or bone substitute. The device is, however, suitable for other highly viscous, tough masses.

### PRIOR ART

Syringe-like containers are frequently used in order to discharge a flowable product to its destination. A piston is present in the container. In order to discharge the product out of the container, the piston is advanced in a distal direction. In the simplest case, the piston is connected to a piston rod which comprises a thumb-rest. In order to discharge the product, the user presses the thumb-rest with his thumb in the distal direction, whilst he holds the container between his index finger and middle finger. However, the discharged amount is not easily able to be controlled in this manner. A relatively large force, which sometimes cannot be applied by the user or can only be applied with difficulty, is additionally required in the case of highly viscous, tough products such as bone cement.

Consequently, instead of providing a piston rod, it is known to provide a pressing-out spindle which is in threaded engagement with a counter piece that is fixed to the container. The piston is then advanced as a result of a helical movement of the pressing-out spindle. As a result, the product can be discharged in a very controlled manner and with little expenditure of force.

However, it is laborious when starting up to pre-screw the pressing-out spindle to begin with until the actual discharging operation starts. It is consequently known to realize the counter piece such that it is able to be moved in a targeted manner into engagement and out of engagement with the pressing-out spindle. As a result, first of all it is possible to advance the pressing-out spindle simply axially without engaging with the counter piece until the force required for this increases significantly in order only then to produce a threaded engagement.

These types of devices are known, for example, from U.S. Pat. No. 4,832,692 or WO 2004/060468. In the case of said devices, the threaded engagement is produced by an actuating lever being pivoted in relation to the container. In U.S. Pat. No. 7,530,970 the threaded engagement is produced as a result of actuating a laterally arranged push button. In the case of the devices of EP 0 565 045 A1 and WO 01/93787 A2, the threaded engagement is produced by an element being rotated relative to the container about the container axis. In the case of the device of U.S. Pat. No. 6,106,496 a frame is displaced relative to the container in order to produce the threaded engagement.

A further device of the named type is provided by Summit Medical Ltd. under the designation “MiniMix™ Precision Delivery Syringe”. In the case of said product, the threaded engagement is produced by an element which is arranged on the container being displaced in the proximal direction, i.e. against the operating direction.

However, the handling of the named devices is not always intuitive. Additionally, at least some of the named devices are constructed in a relatively complicated manner and are correspondingly expensive to produce and are prone to error.

It is also known to produce bone cement by mixing a solid component (powdery in the majority of cases) with a liquid component directly in the syringe-like container. Combined mixing and discharging devices have become known for this purpose where a mixing element is arranged in the container. Such a mixing and discharging device is provided, for example, in WO 2012/174670. In the case of the device disclosed there, the mixing element is connected to a mixing rod which is guided right through the piston to the outside. On its proximal end, the mixing rod is connected to an actuating ring. In order to mix the product, the user takes hold of the actuating ring and with this moves the mixing rod relative to the container. In order then to discharge the product, the user removes the actuating element from the mixing rod and pushes a hollow threaded spindle onto the mixing rod. So that the actuating element is able to be removed from the mixing rod, the mixing rod comprises a predetermined breaking point at which a proximal region of the mixing rod including the actuating ring is able to be broken off. As an alternative to this, the actuating ring is connected to the mixing rod by means of a screw connection and can be unscrewed from the mixing rod.

A disadvantage of a predetermined breaking point is that when the mixing rod is broken off there is a risk of injury. As a rule, the function of the predetermined breaking point is not self-explanatory, which is why training is often necessary. In addition, the predetermined breaking point weakens the mixing rod such that there is the risk of the mixing rod breaking unintentionally even during the mixing process. A disadvantage of a screw connection is that it is laborious and time-consuming to unscrew the actuating ring from the mixing rod. The time required to do this is usually not available in an operating room. In addition, there is the risk of the bone cement starting to harden during this time before it is discharged.

### REPRESENTATION OF THE INVENTION

In a first aspect, the present invention provides a discharging device where a pressing-out spindle with an external thread is first of all able to be displaced axially in relation to a counter piece with an internal thread before the threaded engagement is secured. In this case, the threaded engagement is to be secured in a particularly simple manner, and the operation of the discharging device when securing the threaded engagement is to be particularly intuitive.

Such a discharging device is provided in claim 1. Further embodiments are provided in the dependent claims.

A device for discharging a product is therefore provided, said device comprises:

a container which forms a reservoir for the product;

a piston which delimits the reservoir in a proximal direction and which is displaceable in a distal direction relative to the container in order to discharge the product out of the reservoir;

a pressing-out spindle in order to exert a pressing-out force on the piston in the distal direction, wherein the pressing-out spindle comprises an external thread; and

a counter piece which is arranged on a proximal end region of the container or is formed integrally with the container and is fixed in relation to the container at least in the proximal direction and with reference to rotations, having an internal thread which is configured for the purpose of forming a threaded engagement with the external thread of the pressing-out spindle,

wherein the counter piece comprises one or several threaded segments which are outwardly deflectable in a lateral direction in order to release the threaded engagement, and

wherein the device comprises a securing element which is arranged on the pressing-out spindle and which is pushable onto the counter piece in the distal direction in order to prevent release of the threaded engagement by the securing element impeding the threaded segments from deflecting in a lateral manner.

The discharging device according to the invention makes it possible to secure the threaded engagement between the pressing-out spindle and the counter piece in a very simple manner. As a result of the securing element being arranged on the pressing-out spindle, it is not disturbing the handling of the discharging device before the pressing-out spindle is inserted. The securing element being pushed onto the pressing-out element in the distal direction makes the handling of the device very simple and intuitive. The device is able to be manufactured in a very cost-efficient manner as a result of its simple construction.

Directions are used in this document as follows: The distal direction or more generally the longitudinal direction is defined by the direction of movement of the piston when the product is being discharged. The proximal direction is opposite to the distal direction. A lateral direction is designated as a direction which runs transversely to the longitudinal direction. A lateral direction which runs at an angle of approximately 90° with respect to the longitudinal direction is also designated as a radial direction.

In a preferred manner, each of the threaded segments is configured on an elastically deflectable spring arm which extends with its free end in the proximal direction. However, it is also conceivable, for example, for the threaded segments to be pre-loaded inwardly with a spring force by separate spring elements or the threaded segments to be provided so as to be laterally movable without any action of a spring force.

In a preferred manner, the counter piece forms a linear latching connection with the pressing-out spindle prior to the pushing-on of the securing element such that the pressing-out spindle is insertable into the counter piece in the distal direction by distally directed threaded flanks of the external thread sliding over proximally directed threaded flanks of the internal thread. However, it is also conceivable for the threaded segments of the counter piece to only engage with the external thread of the pressing-out spindle when the securing element is pushed onto the counter piece, and for the pressing-out spindle to be able to be displaced freely beforehand in relation to the counter piece.

In order to ensure a latching connection which allows a movement only in one direction, the external thread can be configured as a buttress thread, having proximally directed threaded flanks which are steeper than the distally directed threaded flanks. In other words, the proximally directed threaded flanks enclose a larger angle with the longitudinal direction than the distally directed threaded flanks. The angle which the distally directed threaded flanks enclose with respect to the longitudinal direction in this case is preferably between 15° and 45°. The angle which the proximally directed threaded flanks enclose with respect to the longitudinal direction is accordingly in a preferred manner at least 60°, in particular in a preferred manner at least 80°, in a particularly preferred manner approximately 90°. The angle can exceed 90° when the thread forms undercuts.

In order to facilitate handling, it is preferred that the securing element is fixable on the pressing-out spindle prior to being pushed onto the counter piece. The fixing can be effected, for example, as a result of a simple frictional connection or as a result of latching-in. When the pressing-out spindle comprises a handle on its proximal end, it is preferred that the securing element is fixable on the pressing-out spindle in the region of the handle. In a preferred manner, the securing element is guided on the pressing-out spindle. Preferably it cannot be removed from the pressing-out spindle whilst the pressing-out spindle is inserted into the counter piece. In a particularly simple embodiment, the securing element is sleeve-shaped and surrounds the pressing-out spindle. The securing element can widen toward its distal end in order to facilitate being pushed onto the counter piece.

The device can be, in particular, a combined mixing and discharging device. For this purpose, the device can additionally comprise:

a mixing element which is movable in the reservoir in order to intermix a product received therein, wherein the mixing element is movable preferably both in the longitudinal direction and in a rotating manner;

a mixing rod which penetrates the piston, wherein the mixing rod comprises a distal end and a proximal end, and wherein the distal end of the mixing rod is connected to the mixing element, and wherein the pressing-out spindle is pushable onto the mixing rod from the proximal end.

In a second aspect, the present invention provides a combined mixing and discharging device where a mixing rod is guided by a piston and an actuating element is arranged releasably on the proximal end of the mixing rod. In this case, the actuating element is able to be removed from the mixing rod in a particularly secure, simple and intuitive manner.

For this purpose a device for mixing and discharging a product is provided, which device comprises:

a container which forms a reservoir for the product;

a piston which delimits the reservoir in a proximal direction and which is displaceable in a distal direction relative to the container in order to discharge the product out of the reservoir;

a mixing element which is movable in the reservoir in order to intermix a product received therein;

a mixing rod which penetrates the piston, wherein the mixing rod comprises a distal end and a proximal end, and wherein the distal end of the mixing rod is connected to the mixing element; and

an actuating element which is connected to the proximal end of the mixing rod by means of a releasable positive-locking or frictional connection and is removable from the mixing rod as a result of releasing the positive-locking or frictional connection, as well as

a locking element which is movable on the actuating element between a locking position and a release position, wherein the locking element prevents release of the positive-locking or frictional connection in the locking position, whilst in the release position it enables release of the positive-locking connection.

In the locking position the locking element therefore secures a positive-locking or frictional connection between the mixing rod and the actuating element. The actuating element can be released from the mixing rod as a result of simply moving the locking element into the release position and releasing the positive-locking or frictional connection. This means it is no longer necessary to break off the mixing rod, with all the disadvantages linked thereto. Compared to a threaded connection, the handling is made a lot easier.

In preferred embodiments, the actuating element comprises one or several connecting structures which form the releasable positive-locking or frictional connection with the mixing rod, wherein the connecting structures are outwardly deflectable in a lateral direction in relation to the mixing rod in order to release the positive-locking or frictional connection. In the locking position then, the locking element impedes the connecting structures from deflecting outward in a lateral manner. In particular, the connecting structures can include one or several elastic spring legs which are deflectable outwardly in a lateral manner and extend in the distal direction along the mixing rod. This makes possible a particularly simple and elegant connection between the mixing rod and the actuating element.

In order to produce a positive-locking connection, it is possible to provide a latching lug which is directed inward in a lateral manner on at least one of the connecting structures. In this case, in a preferred manner the mixing rod comprises one or several lateral latching indentations, wherein in each case a positive-locking connection is formed by one latching lug and one latching indentation. As an alternative to this, however, it is also conceivable, for example, for only a frictional connection, which is secured by the locking element, to exist between the connecting structures and the proximal end of the mixing rod.

In a particularly simple embodiment, the locking element forms a sleeve which, in the locking position, rests laterally on the connecting structures in order to prevent the connecting structures from deflecting laterally, and which, in the release position, enables the connecting structures to deflect laterally.

In a preferred manner, the locking element is displaceable relative to the actuating element from the locking position into the release position by a movement in the proximal direction. This results in a particularly intuitive operation when the actuating element is to be removed as the actuating element together with the locking element is then also removed in the proximal direction.

The actuating element can comprise a handle region which is configured for the purpose of being gripped by a hand of a user (for example in the form of a classic handle) and/or for receiving at least one finger of the hand (for example in the form of a ring). In particular, the handle region can comprise the form of a ring, the size of which is chosen such that it is suitable to receive at least one finger of the hand, in particular the index finger. The locking element is then preferably arranged distally from the handle region on the actuating element. The locking element can comprise a lateral flange region which is arranged such that it is possible for the user to pull the locking element on the flange region in the proximal direction toward the handle region of the actuating element by way of one or several fingers of the same hand without releasing the handle region.

In order to prevent unintentional release of the actuating element, it is preferred when, in the locking position, the locking element is held on the actuating element as a result of a releasable frictional or positive-locking connection, wherein the releasable frictional or positive-locking connection is releasable by overcoming an increased minimum force which acts in the direction of the release position.

In order to facilitate the discharging of the product once the actuating element has been removed, the device can additionally comprise:

a pressing-out spindle in order to exert a pressing-out force on the piston in the distal direction, wherein the pressing-out spindle comprises an external thread and is pushable onto the mixing rod from the proximal end of the mixing rod once the actuating element has been removed from the mixing rod; and

a counter piece which is arranged on a proximal end region of the container or is formed integrally with the container and is fixed in relation to the container at least in the proximal direction and with reference to rotations, having an internal thread which forms a threaded engagement with the external thread of the pressing-out spindle.

In order to facilitate the subsequent pushing-on of a pressing-out element, it is preferred when the mixing rod does not widen at its proximal end.

In a third aspect, the present invention provides a discharging device where the contents of the device are protected particularly well against heating up as a result of the hand heat of a user.

For this purpose, a device for discharging a product is provided which comprises: a container which comprises a peripheral wall which delimits a reservoir for the product;

a piston which delimits the reservoir in a proximal direction and which is displaceable in a distal direction relative to the container in order to discharge the product from the reservoir, wherein on the outside the container comprises insulating projections which protrude from the peripheral wall in order to reduce thermal contact with the interior of the container when a user takes hold of the container by the peripheral wall.

The insulating projections can include in particular insulating ribs. In a preferred manner these extend along a longitudinal direction of the container.

The first, second and third aspects of the invention can be realized independently of one another or in arbitrary combination with one another.

### BRIEF DESCRIPTION OF THE DRAWINGS

Preferred embodiments of the invention are described below by way of the drawings which serve purely for explanation and are not to be interpreted in a restricting manner, in which drawings:

FIG. 1 shows a perspective view of a mixing and discharging device according to a preferred exemplary embodiment of the present invention;

FIG. 2 shows a side view of parts of the mixing and discharging device of FIG. 1;

FIG. 3 shows a central longitudinal section of the parts of FIG. 2;

FIGS. 4-7 show a central longitudinal section of parts of the mixing and discharging device of FIG. 1 in different stages of use;

FIG. 8 shows a central longitudinal section of a view of a detail of FIG. 4; and

FIG. 9 shows a central longitudinal section of a view of a detail of FIG. 5.

### DESCRIPTION OF PREFERRED EMBODIMENTS

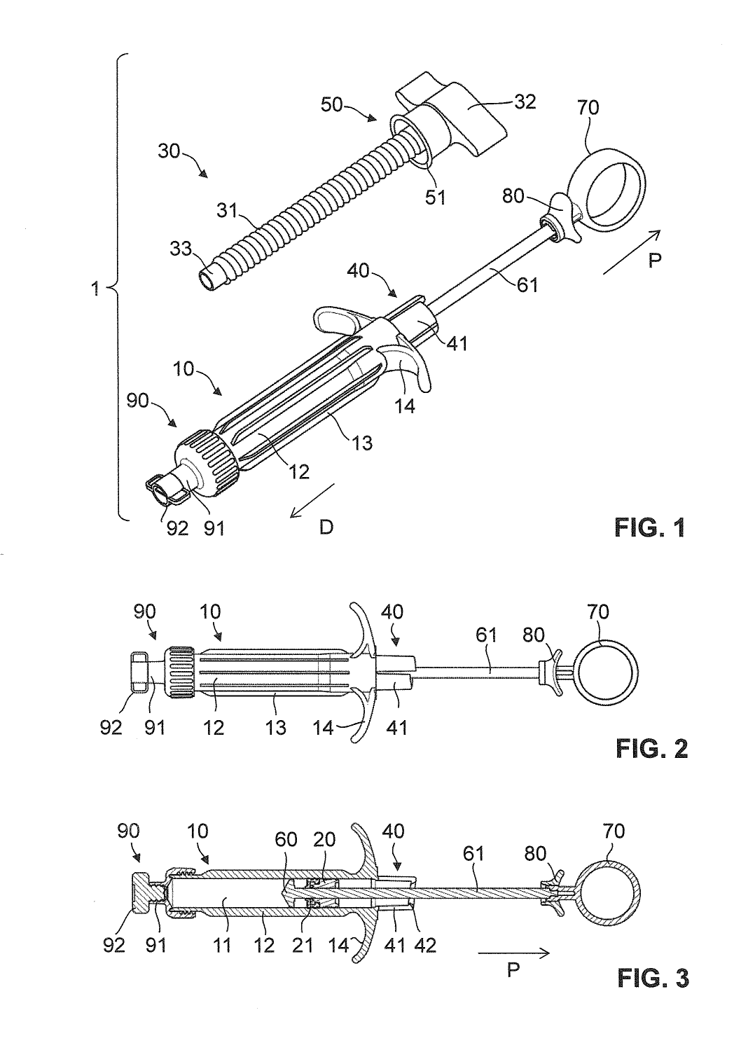

FIG. 1 shows a preferred exemplary embodiment of a mixing and discharging device 1 according to the present invention. FIGS. 2 and 3 show a side view and a central longitudinal section of parts of said mixing and discharging device.

The mixing and discharging device 1 comprises a container 10 which forms a syringe body. With its peripheral wall 12, the container 10 delimits a cylindrical reservoir 11 which defines a longitudinal direction by way of its cylinder axis. Insulating ribs 13 which extend parallel to the longitudinal direction are configured on the outside surface of the peripheral wall 12. Said insulating ribs 13 serve for the purpose of reducing the transmission of body heat to the peripheral wall 12 when a user takes hold of the container 10 with his fingers. As a result, a product that is accommodated in the reservoir 11 is protected from excessive heating. Two oppositely situated lateral holding flanges 14 are formed in a proximal end region on the container 10 in order to hold the container 10 between the index finger and middle finger of one hand of the user.

A piston 20 is arranged in the container 10 so as to be displaceable along the longitudinal direction, the piston 20 abutting in a sealing manner with a sealing element 21 on its circumference against the inside surface of the peripheral wall 12. As a result, the piston 20 defines the reservoir 11 in a fluid-tight manner in a proximal direction P.

At its distal end, the container 10 comprises a distal removal opening, the size of which is such that the cross section of the reservoir 11 is not tapered at the distal end. An external thread, onto which a closure 90 is screwed, is formed on the peripheral wall 12 of the container 10 in the region of the removal opening. The closure 90 comprises a discharging spout 91, the cross section of which is smaller than the cross section of the reservoir 11. The discharging spout 91 is initially closed by a screw-connected sealing plug 92.

A mixing element 60 is arranged in the reservoir 11 between the piston 20 and the closure 90. The mixing element 60 is configured in a manner that is known per se. It can comprise several arms, for example, which extend radially outward proceeding from a central hub and are connected to short arcuate segments at their free ends. The mixing element 60 is connected to a mixing rod 61. In the present example, the mixing element 60 is formed integrally with the mixing rod 61; obviously, however, it is also conceivable for the mixing element 60 and the mixing rod 61 to be separate parts which are connected together, for example, by a screw-type or latching connection. The piston 20 comprises a central longitudinal bore, and the mixing rod 61 extends right through said longitudinal bore. As a result, the mixing rod 61 penetrates the piston 60 in the axial direction. At its proximal end, the mixing rod 61 is connected in a positive-locking but releasable manner to a ring-shaped actuating element 70. The manner in which the positive-locking connection is produced and released will be described in more detail below. A locking element 80 prevents the positive-locking connection between the mixing rod 61 and the actuating element 70 being unintentionally released.

The mixing and discharging device 1 additionally includes a hollow pressing-out spindle 30 as a separate element which is provided with an external thread 31. At its proximal end, the pressing-out spindle 30 is provided with a handle 32 which serves for the purpose of rotating the pressing-out spindle about its longitudinal axis. At its distal end, the pressing-out spindle 30 comprises a distal end region 33 which comprises an outside diameter that is smaller than that of the external thread 31.

A counter piece 40 for the pressing-out spindle 30 is arranged at the proximal end of the container 10. In the present example, the counter piece 40 is formed integrally with the container 10; the counter piece 40 can also be manufactured as a separate piece and be connected subsequently to the container 10. The counter piece 40 has the form of a threaded sleeve which is divided into segments by several axially extending incisions. Each of the segments forms an elastically outwardly deflectable spring arm 41, on the proximal free end of which an inwardly pointing threaded segment 42 is formed.

The mixing and discharging device 1 additionally comprises a securing sleeve 50 which is arranged on the pressing-out spindle 30 in the region of the handle 32. The function of the securing sleeve 50 will be explained in more detail below. The securing sleeve 50 widens slightly in its distal end region 51. The securing sleeve 50 is frictionally connected to the pressing-out spindle 30 on its proximal end and, as a result, is held on the pressing-out spindle 30 so as to be releasable.

Initially, the mixing and discharging device 1 is situated in the state in FIGS. 1-3. Beforehand, two components of a product, which are subsequently to be mixed together, have been filled into the reservoir 11. To this end, the user takes hold of the actuating element 70 and with this moves the mixing rod 61 in the axial direction and at the same time back and forth in the circumferential direction. As a result, the mixing element 60 is moved in a corresponding manner in the reservoir 11 and thus mixes the components.

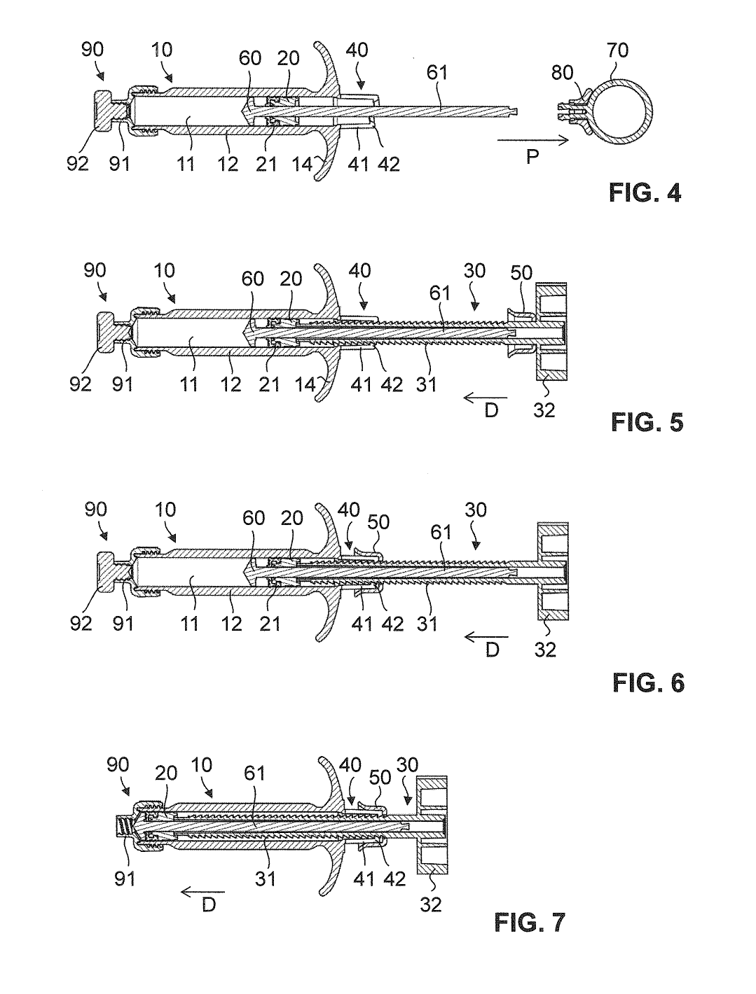

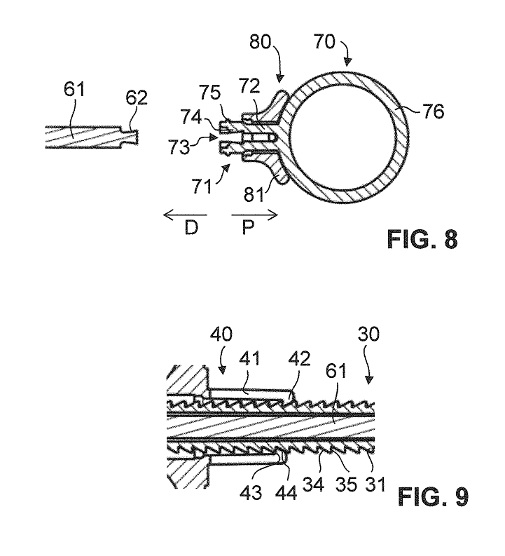

In order to discharge the finished product, the user first of all removes the actuating element 70, as is shown in FIG. 4. To this end, the user inserts his thumb into the ring-shaped actuating element 70 and pulls the locking element 80 with the index finger and middle finger in the proximal direction P. As a result, the connection between the mixing rod 61 and the actuating element 70 is released, as will be explained in more detail below in conjunction with FIG. 8.

The user then pushes the pressing-out spindle 30 in the distal direction D onto the mixing rod 61. In this case, the distal end region 33 of the pressing-out spindle 30 first of all passes into the region of the counter piece 40. On account of its reduced diameter, the distal end region 33 can be easily pushed between the threaded segments 42 until the external thread 31 of the pressing-out spindle 30 contacts the threaded segments 42. The distally directed threaded flanks of the external thread 31 now slide over the proximally directed threaded flanks of the threaded segments 42 and as a result deflect the threaded segments 42 radially outward. Accordingly, the spring arms 41 spring elastically outward. As soon as the threaded segments 42 engage with the external thread 31, the spring arms 41 spring back inward again. In all, a linear latching connection between the pressing-out spindle 30 and the counter piece 40 is formed in this manner.

The user then advances the pressing-out spindle 30 axially in the distal direction D until the pressing-out spindle 30 abuts against the proximal end of the piston 20 with its distal end region 33. This state is shown in FIG. 5. From this moment, a significantly increased counter force acts against the pressing-out spindle 30 advancing any further in a linear manner. The linear latching connection between the counter piece 40 and the pressing-out spindle 30 prevents the pressing-out spindle 30 from being able to be pulled back.

In order to secure the threaded connection between the pressing-out spindle 30 and the counter piece 40, the user now pushes the securing sleeve 50 on the pressing-out spindle 30 forward in the distal direction D until the securing sleeve 50 laterally encompasses the counter piece 40. This is shown in FIG. 6. In this case, the widened distal end region of the securing sleeve 50 facilitates the pushing of the securing sleeve 50 onto the counter piece 40. The securing sleeve 50 then rests laterally on the spring arms 41 and, as a result, prevents the spring arms 41 being able to continue to be deflected outward.

In order to press the product out of the reservoir 11, the user first of all removes the sealing plug 92. The user then rotates the threaded spindle 30 in the clockwise direction by the handle 32 and thus screws the threaded spindle 30 into the container 10 in the distal direction D. As a result, the piston 20 is advanced in the distal direction D and presses the product right through the discharging spout 91 out of the reservoir 11. The state at the end of the discharging operation is shown in FIG. 7.

The connection between the piston rod 61 and the actuating element 70 is now explained in more detail by way of FIG. 8. The fastening element 70 comprises an actuating region in the form of a ring 76 and a fastening region 71 which extends in the distal direction D from the ring 76. The fastening region 71 comprises two radially opposite connecting structures in the form of spring legs 72 which, proceeding from the ring 76, extend in the distal direction D parallel to the longitudinal axis. Each of the two springs legs comprises a latching lug 74 on the inside. The spring legs 72 together delimit a gap 73 into which the proximal free end of the mixing rod 61 is insertable.

The mixing rod 61 comprises at its proximal free end two oppositely situated latching indentations 62 which are complementary to the latching lugs 74 of the fastening region 71. When the free end of the mixing rod 61 is pushed into the gap 73, the spring legs 72 spring outward until the latching lugs 74 engage in the latching indentations 62 and thus produce a positive-locking connection between the mixing rod 61 and the actuating element 70. In this case, the positive-locking connection acts both in the axial direction and the circumferential direction, i.e. the actuating element 70 is fixed on the mixing rod 61 both with reference to axial movements and with reference to rotations about the longitudinal axis.

Said positive-locking connection is secured by the sleeve-like locking element 80. FIGS. 1-3 show the locking element 80 situated in a locking position in which it prevents the spring legs 72 from deflecting in a lateral manner. As a result, the locking element 80 prevents the positive-locking connection between the mixing rod 61 and the actuating element 70 being released. In the locking position, the locking element is fixed in a frictional locking manner on the actuating element 70 by means of a radially outwardly protruding holding bead 75. In FIG. 8, the locking element 80, in contrast, is situated in a release position in which it makes it possible for the spring legs 72 with the latching lugs 74 to deflect in a lateral manner and consequently for the positive-locking connection to be released, by the actuating element 70 being removed from the mixing rod 61 in the proximal direction P. In order to be able to move the locking element 80 into the release position in a simple and intuitive manner, the locking element 80 comprises oppositely situated flange regions 81 which are configured for the purpose of being pulled in the proximal direction by means of the index and middle fingers of one hand, the index finger of which is situated in the ring 76. On account of the holding bead 75, a certain minimum resistance has first of all to be overcome before the locking element 80 can be moved into the release position.

FIG. 9 shows an enlarged view of the connection between the pressing-out spindle 30 and the counter piece 40. As has already been explained above, the counter piece 40 forms a sleeve which is subdivided by axial incisions into several sleeve segments which act as spring arms 41. On its free end, each of the spring arms 41 comprises a threaded segment 42. The threaded segment comprises a steep distal flank 43, which forms an angle of 90° to the longitudinal direction, as well as a flat proximal flank 44, which forms an angle of approximately 30° to the longitudinal direction. The flanks of the external thread 31 on the outside circumference of the pressing-out spindle 30 are configured in a complementary manner hereto. The distally directed flanks 34 are configured correspondingly flatter than the proximally directed flanks 35. In this way, the pressing-out spindle 30 can be advanced axially in the distal direction in relation to the counter piece 40. In this case, the external thread 31 and the threaded segments 42 form a linear latching connection where the flat distally directed flanks 34 of the external thread 31 slide over the flat proximally directed flanks 44 of the threaded segments 42, whilst the steep proximally directed flanks 35 of the external thread 31 together with the steep distally directed flanks 43 of the threaded segments 42 prevent the pressing-out spindle 30 from being pulled back.

Diverse modifications are obviously possible without departing from the scope of the present invention. In particular, the combination shown here of pressing-out spindle 30, counter piece 40 and securing sleeve 50 can also be used in the case of a discharging device without a mixing element 60 and mixing rod 61. Conversely, the manner in which the actuating element 70 is held on the mixing rod 61 by means of a locking element 80 can be used independently of a certain pressing-out element. The individual elements can also be designed differently to how they are shown here as an example. Thus, for example, the pressing-out spindle 30 can comprise a differently designed handle and can be formed differently at its distal end. Instead of a single-start thread, a double-start or multiple-start thread is also conceivable. The counter piece 40 can also be designed differently. Thus, the counter piece does not forcibly have to be sleeve-shaped, and not all the segments of the counter piece need to form spring arms. The securing sleeve 50 can comprise a form other than that shown here as long as it is suitable to impede parts of the counter piece 40 from deflecting outward. The container 10 does not forcibly need to comprise insulating ribs. If insulation is desired, other types of insulating structures can also be present on the container wall 12. Insulating projections can also be advantageous in the case of other types of syringe-like containers. The container can also be designed differently on its distal end to as shown here and in particular can be provided with a different type of closure.

### LIST OF REFERENCES

1 Mixing and discharging device
10 Container
11 Reservoir
12 Peripheral wall
13 Insulating rib
14 Holding flange
20 Piston
21 Sealing element
30 Pressing-out spindle
31 External thread
32 Handle
33 Distal end region
34 Distally directed flank
35 Proximally directed flank
40 Counter piece
41 Spring arm
42 Threaded segment
43 Distally directed flank
44 Proximally directed flank
50 Securing sleeve
51 Distal end region
60 Mixing element
61 Mixing rod
62 Latching indentation
70 Actuating element
71 Fastening region
72 Spring leg
73 Gap
74 Latching lug
75 Holding bead
76 Ring
80 Locking element
81 Flange region
90 Closure
91 Discharging spout
92 Sealing plug

1. A device for discharging a product, said device comprising:
a container which forms a reservoir for the product;
a piston which delimits the reservoir in a proximal direction and which is displaceable in a distal direction relative to the container in order to discharge the product out of the reservoir;
a pressing-out spindle in order to exert a pressing-out force on the piston in the distal direction, wherein the pressing-out spindle comprises an external thread; and
a counter piece which is arranged on a proximal end region of the container or is formed integrally with the container and is fixed in relation to the container at least in the proximal direction and with reference to rotations, having an internal thread which is configured for the purpose of forming a threaded engagement with the external thread of the pressing-out spindle,
wherein the counter piece comprises one or several threaded segments which are outwardly deflectable in a lateral direction in order to release the threaded engagement, and
wherein the device comprises a securing element which is arranged on the pressing-out spindle and which is pushable onto the counter piece in the distal direction in order to prevent release of the threaded engagement by the securing element impeding the threaded segments from deflecting in a lateral manner.

2. The device as claimed in claim 1, wherein each of the threaded segments is configured on an elastically deflectable spring arm which extends with its free end in the proximal direction.

3. The device as claimed in claim 1, wherein the counter piece 44-forms a linear latching connection with the pressing-out spindle prior to the pushing on of the securing element such that the pressing-out spindle is pushable into the counter piece in the distal direction by distally directed threaded flanks of the external thread sliding over proximally directed threaded flanks of the internal thread.

4. The device as claimed in claim 1, wherein the external thread is configured as a buttress thread, having distally directed threaded flanks and proximally directed threaded flanks, wherein the proximally directed threaded flanks are steeper than the distally directed threaded flanks.

5. The device as claimed in claim 1, wherein the securing element is fixable on the pressing-out spindle prior to being pushed onto the counter piece.

6. The device as claimed in claim 5, wherein the pressing-out spindle comprises a handle on its proximal end, and wherein the securing element is fixable on the pressing-out spindle in the region of the handle prior to being pushed onto the counter piece.

7. The device as claimed in claim 1, wherein the securing element is guided on the pressing-out spindle.

8. The device as claimed in claim 1, wherein the securing element cannot be removed from the pressing-out spindle whilst the pressing-out spindle is inserted into the counter piece.

9. The device as claimed in claim 1, wherein the securing clement is sleeve-shaped.

10. The device as claimed in claim 9, wherein the securing element widens toward its distal end.

11. The device as claimed in claim 1, which device additionally comprises:
a mixing element which is movable in the reservoir in order to intermix a product received therein;
a mixing rod which penetrates the piston wherein the mixing rod comprises a distal end and a proximal end, and wherein the distal end of the mixing rod is connected to the mixing element, wherein the pressing-out spindle is pushable onto the mixing rod from the proximal end.

12. The device as claimed in claim 11, which device additionally comprises:
an actuating element which is connectable to the proximal end of the mixing rod by means of a releasable positive-locking or frictional connection and, once the positive-locking or frictional connection has been released, is removable from the mixing rod in order to push the pressing-out spindle onto the mixing rod.

13. A device for mixing and discharging a product, said device comprising:
a container which forms a reservoir for the product;
a piston which delimits the reservoir in a proximal direction and which is displaceable in a distal direction relative to the container in order to discharge the product out of the reservoir;
a mixing element which is movable in the reservoir in order to intermix a product received therein;
a mixing rod which penetrates the piston, wherein the mixing rod comprises a distal end and a proximal end, and wherein the distal end of the mixing rod is connected to the mixing element; and
an actuating element which is connected to the proximal end of the mixing rod by means of a releasable positive-locking or frictional connection, wherein the device additionally comprises a locking element which is movable on the actuating element between a locking position and a release position, wherein the locking element prevents release of the positive-locking or frictional connection in the locking position whilst in the release position it enables release of the positive-locking or frictional connection.

14. The device as claimed in claim 13, wherein the actuating element comprises one or several connecting structures which form with the mixing rod the releasable positive-locking or frictional connection,
wherein the connecting structures are outwardly deflectable in a lateral direction in relation to the mixing rod in order to release the positive-locking or frictional connection, and
wherein in the locking position the locking element impedes the connecting structures from deflecting outward in a lateral manner.

15. The device as claimed in claim 14, wherein the connecting structures include one or several elastic spring legs which are deflectable outward in a lateral manner and extend in the distal direction along the mixing rod.

16. The device as claimed in claim 14, wherein a latching lug which is directed inward in a lateral manner is configured on at least one of the connecting structures and wherein the mixing rod comprises one or several lateral latching indentations wherein in each case a positive-locking connection is formed by one latching lug and one latching indentation.

17. The device as claimed in claim 14, wherein the locking element forms a sleeve which, in the locking position, rests laterally on the connecting structures in order to prevent the connecting structures from deflecting in a lateral manner, and which, in the release position. enables the connecting structures to deflect in a lateral manner.

18. The device as claimed in claim 13, wherein the locking element is displaceable relative to the actuating element from the locking position into the release position by a movement in the proximal direction.

19. The device as claimed in claim 18, wherein the actuating element comprises a handle region which is configured for the purpose of being gripped by a hand of a user and/or receiving at least one finger of the hand, in particular the index finger,
wherein the locking element is arranged on the actuating element distally from the handle region, and
wherein the locking element comprises a lateral flange region which is arranged such that it is possible for the user to pull the locking element on the flange region in the proximal direction toward the handle region of the actuating element by way of one or several fingers of the same hand without releasing the handle region.

20. The device as claimed in claim 19, wherein the handle region comprises the form of a ring, the size of which is chosen such that it is suitable to receive at least one finger of the hand, in particular the index finger.

21. The device as claimed in claim 13, wherein, in the locking position, the locking element is held on the actuating element as a result of a releasable frictional or positive-locking connection, and wherein the releasable frictional or positive-locking connection is releasable by overcoming a minimum resistance which acts in the direction of the release position.

22. The device as claimed in claim 13, which device additionally comprises:
a pressing-out spindle in order to exert a pressing-out force on the piston in the distal direction, wherein the pressing-out spindle comprises an external thread and is pushable onto the mixing rod from the proximal end of the mixing rod 00-once the actuating element has been removed from the mixing rod; and
a counter piece which is arranged on a proximal end region of the container or is formed integrally with the container and is fixed in relation to the container at least in the proximal direction and with reference to rotations, having an internal thread which forms a threaded engagement with the external thread of the pressing-out spindle.

23. The device as claimed in claim 13, wherein the mixing rod does not widen at its proximal end.

24. A device for discharging a product, said device comprising:
a container which comprises a peripheral wall which delimits a reservoir for the product;
a piston which delimits the reservoir in a proximal direction and which is displaceable in a distal direction relative to the container in order to discharge the product out of the reservoir, wherein on the outside, the container comprises insulating projections which protrude from the peripheral wall in order to reduce thermal contact with the interior of the container when a user takes hold of the container by the peripheral wall.

25. The device as claimed in claim 24, wherein the insulating projections include insulating ribs.

26. The device as claimed in claim 25, wherein the insulating ribs extend along a longitudinal direction of the container.
